(19) United States
(12) Patent Application Publication
(10) Pub. No.: US 2014/0091125 A1
(43) Pub. Date: Apr. 3, 2014

(54) ROD-SHORTENING TOOL

(71) Applicant: Steffen DIRR, Voehringen (DE)

(72) Inventor: Steffen DIRR, Voehringen (DE)

(21) Appl. No.: 13/678,182

(22) Filed: Nov. 15, 2012

(30) Foreign Application Priority Data
Oct. 2, 2012 (DE) 102012109340.7

(51) Int. Cl. A61B 17/88; B26F 3/00; B26D 3/08

(52) U.S. Cl. CPC A61B 17/8863; B26D 3/08; B26F 3/002
USPC 225/2; 30/94; 225/96; 30/102; 83/880

(57) ABSTRACT

A tool for shortening an implant rod has a base housing part and a movable housing part shiftable toward and away from each other in a predetermined direction. At least one guide roller is provided on the movable housing part, and a cutter is provided on the base housing part. The rod is fittable between the cutter and the guide roller in a spread position of the housing parts. A biasing unit urges the parts together and thereby presses the cutter into the rod with a predetermined force. Thus the cutter is pressed against the workpiece with a force that does not change and that is not under the control of the user, ensuring that the force applied lies within an acceptable safe range.

### FIELD OF THE INVENTION

The present invention relates to tool for shortening a rod used in a spinal implant. More particularly this invention concerns method of and tool for doing this.

### BACKGROUND OF THE INVENTION

Spinal surgery, in particular, entails the need to join multiple implants to each other. To this end, pedicle screws are inserted first into individual vertebrae and interconnected by a rod. To accomplish this, however, the length of the rod must be adapted to the specific requirements of the patient. As a result, it is thus necessary to shorten the rod to the appropriate length. This is generally done using ordinary pliers to cut to length the rod that is to be shorted. However, this often results in the cut surface of the rod having sharp edges, with the result that there is a risk of injuring soft tissues of the patient. There is also the danger that the section of the rod that is to be cut to length will twist uncontrolledly.

### OBJECTS OF THE INVENTION

It is therefore an object of the present invention to provide an improved rod-cutting tool.

Another object is the provision of such an improved rod-cutting tool and method of cutting a rod that overcomes the above-given disadvantages, in particular that cuts the rod cleanly without bending it.

### SUMMARY OF THE INVENTION

A tool for shortening an implant rod has according to the invention a base housing part and a movable housing part shiftable toward and away from each other in a predetermined direction. At least one guide roller is provided on the movable housing part, and a cutter is provided on the base housing part. The rod is fittable between the cutter and the guide roller in a spread position of the housing parts. A biasing unit urges the parts together and thereby presses the cutter into the rod with a predetermined force. Thus the cutter is pressed against the workpiece with a force that does not change and that is not under the control of the user, ensuring that the force applied lies within an acceptable safe range.

Unlike tools already known for shortening an implant, the tool according to the invention no longer necessitates severing the entire implant with a cutting element. Instead, all that is required is to generate a predetermined score line, which approach has a positive benefit in terms of the required force to be applied and the time taken to cut the implant rod to length. The biasing unit furthermore ensures that no resetting of the cutting element is required, and this aspect too has a positive benefit in terms of the ability to manipulate the tool.

It has furthermore been found advantageous for the biasing unit to be composed of an elastic return element, in particular, a compression spring, that is preferably braced between the housing parts. The use of a return element of this type, which can for example be a compression spring, enables an essentially maintenance-free constructive design to be implemented. However, provision can also be made according to the invention whereby the elastic return element is a tension spring.

It has furthermore been found especially advantageous to provide an eccentric lever that can be adjusted between a biasing position and a release position, and is preferably associated with the movable housing part, the lever being used to work the biasing unit. Adjusting the biasing unit with an eccentric lever ensures that only an extremely small force needs to be applied to operate the tool due to the mechanical advantage of the eccentric lever.

It has furthermore been found advantageous to provide a connecting rod to transfer the spring-loading movement of the eccentric lever to the biasing unit. This then provides a simple means of transferring the motion of the eccentric lever to the biasing unit in order to adjust the cutting element against the implant to be cut and to continuously maintain the force applied to the implant.

It has furthermore been found advantageous if the position of the eccentric lever is able to be fixed. Fixing the position of the eccentric lever in the tensioning position ensures that the lever cannot unintentionally release, with the result that the need for continuous resetting is eliminated.

It has furthermore been found especially advantageous to provide at least one breaking aid that is preferably associated with the housing. This then enables the implant to be broken with a minimal application of force after generating the predetermined score line.

It has furthermore been found especially advantageous for the breaking aid to be a bore or hole whose diameter corresponds at least to the diameter of the implant rod to be broken. As a result, after the score line has been formed the implant rod can be inserted into the hole and the protruding end simply snapped off. An advantageous approach is to allow the portion of the implant to be cut off to protrude from the breaking aid and then act only on this portion in order to prevent any deformation of the part of implant rod that is going to be used.

It has furthermore been found advantageous to associate with the breaking aid a position indicator that interacts with a predetermined score line of the implant, the indicator fixing the position of the implant. A position indicator of this type, which can be provided, for example, in the form of a spherical seat, facilitates shortening the implant since the implant is then located in a well-defined position after insertion into the breaking aid.

It has furthermore been found advantageous for a handle to be provided on the housing. This handle first of all assists in generating the predetermined score line since it facilitates rotating the tool around the implant to generate the predetermined score line. In addition, a handle of this type also assists in breaking the implant that is provided here with an appropriate lever.

It has been found especially advantageous for the is cutting element to be composed of a cutting roller. This design of the cutting element enables the predetermined score line to be formed around the entire outer circumference of the implant. However, provision is also made within the scope of the invention whereby the cutting element is in the form of a cutting edge that generates the predetermined score line by cutting.

The invention furthermore comprises a method for producing an individually adapted implant by using a predetermined score line that is generated by a tool that includes at least one guide roller and a cutting element, where the cutting element is continuously reset and biased against the implant by a biasing unit. This method provides the advantage that the need is eliminated to reset the cutting element as the predetermined score line becomes deeper, which aspect has a positive effect on user friendliness.

An approach has furthermore been found especially advantageous in terms of manipulation whereby adjustment of an eccentric lever is used to adjust the tool between a release position suitable for inserting and removing the implant, and a tensioning position generating the predetermined score line. Operating the tool with an eccentric lever ensures that the applied force required to generate the predetermined score line is reduced since the lever action of the eccentric lever can be utilized.

It has furthermore been found advantageous to generate the predetermined score line on the implant by relative rotation between the implant and the tool. This enables the predetermined score line to be generated around the complete outer circumference of the implant, thereby facilitating cutting the implant to length and ensuring a clean end on the implant rod after shortening.

It has furthermore been found advantageous if the implant is shortened by a breaking aid provided on the tool after the predetermined score line has been generated. After the predetermined score line is generated, the implant can thus be removed from the tool and inserted into the breaking-aid hole. Following this, the implant can simply be snapped off at the predetermined score line and thereby sized to the desired length.

### BRIEF DESCRIPTION OF THE DRAWING

The above and other objects, features, and advantages will become more readily apparent from the following description, reference being made to the accompanying drawing in which:

FIG. 1 is a side view partly in vertical section of a tool according to the invention;

FIG. 2 is a side view of the tool according to the invention with inserted implant rod; and

FIG. 3 shows the tool with the eccentric lever in different positions.

### DETAILED DESCRIPTION

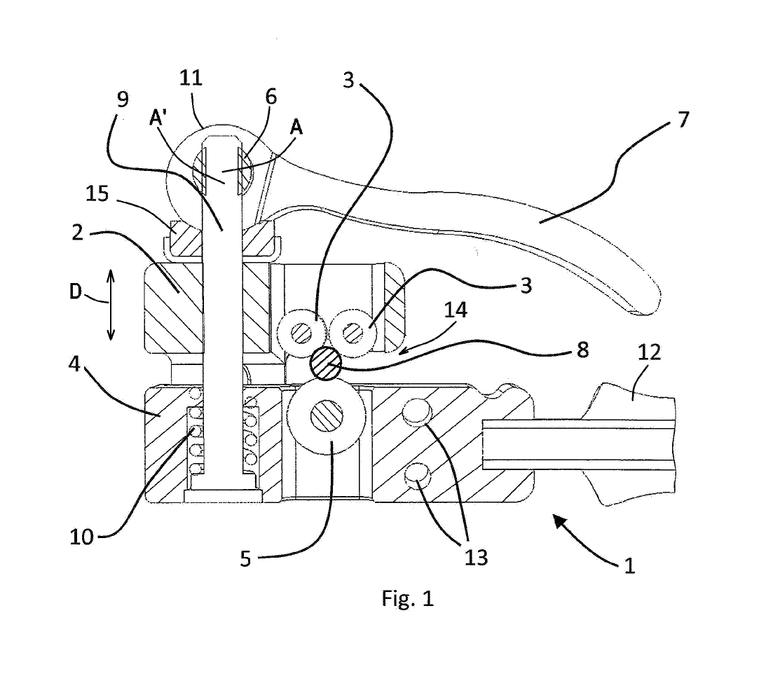

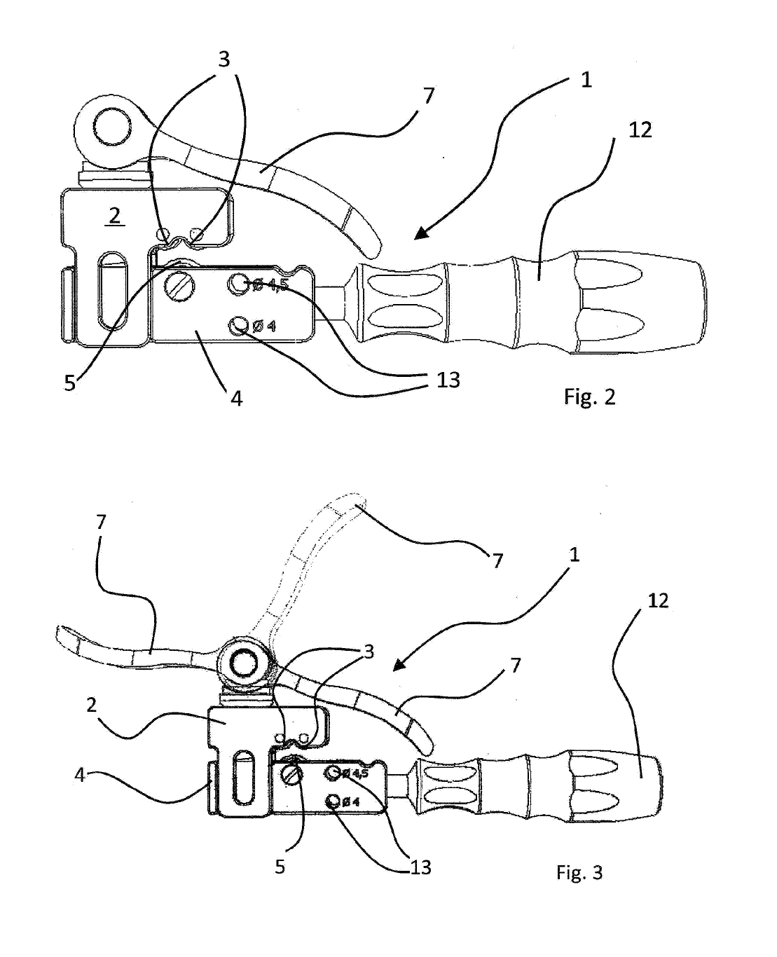

As seen in FIG. 1 a tool 1 for shortening an implant rod 8 by forming a score line in the rod 8. The tool 1 has a housing with a movable part 2 that carries two guide rollers 3 rotatable about respective parallel axes and a base part 4 in which is a cutter wheel 5 with a sharp edge is rotatable about an axis parallel to that of the guide rollers 3. The parts 2 and 4 can move limitedly relative to each other in a direction D to shift the roller 4 toward and away from the rollers 3.

A lever 7 is pivotal about an axis A relative to the housing 2, 4 and serves for shifting the parts 2 and 4 toward each other. To this end a pivot pin 6 on the axis is set in one end of the lever 7 and is coupled to a connecting rod 9 that extends in the direction D through the part 2 and mostly through the part 4. Inside the part 4 a tension spring 10 bears downward on a head of the rod 9 and upward on the part 4 so as to urge the two parts 2 and 4 together in the direction D. The one end of the lever 11 has a cylindrical outer surface centered on an axis A′ offset from the pivot axis A along the rod and fitted to a shoe 15 bearing downward on the part 2. Thus pivoting the lever 7 about the axis A from the left-hand solid-line position of FIG. 3 through the dashed-line position to the right-hand solid-line position of FIG. 3 will shift the rod 6 upward in the direction D relative to the parts 2 and 4. This will compress the spring 10 against the part 4 and urge it upward.

The part 4 is provided with a handle 12 extending perpendicularly to the direction D and is formed with a pair of labeled cylindrical holes 13 dimensioned to snugly receive distraction rods of standard diameters.

To shorten the rod 8, the lever 7 is moved into the position extending from the axis A away from the handle 12 to move the parts 2 and 4 apart. The rod 8 is then shifted into the mouth 14 and set between the rollers 3 and 5.

Then the lever 7 is pivoted through about 180° to a position extending along the handle 7. This presses the cutter wheel 4 with a predetermined pressure against the rod 8 while pressing this rod 8 against the guide rollers 3. Then, while the rod 8 is prevented from rotating by, for instance, gripping it offset from the tool 1 with pliers, the entire tool 1 is rotated one or more turns around the rod 8. The cutter 5, is continuously pressed by the spring 10 into the rod 8 so that with each rotation it will sink somewhat deeper into this rod 8, forming an annular circumferential groove in it.

The lever 7 is then pivoted back away from the handle 12 and the rod 8 is taken out from between the rollers 3 and 5 and is pushed into whichever of the holes 13 it more fits most snugly. The score line formed by the cutter 5 is positioned at the side face of the tool 1 and the projecting part is bent to the side, cleanly fracturing the rod 8 at the score line. about the axis 7. Normally the part of the rod 8 that projects from the tool 1 for snapping off is the scrap part, so that the part that goes into the patient is not bent or damaged.

I claim:

1. A tool for shortening an implant rod, the tool comprising:
a base housing part and a movable housing part shiftable toward and away from each other in a predetermined direction;
at least one guide roller on the movable housing part;
a cutter on the base housing part, the rod being fittable between the cutter and the guide roller in a spread position of the housing parts; and
biasing means for urging the parts together and thereby pressing the cutter into the rod with a predetermined force.

2. The rod-shortening tool defined in claim 1, wherein the biasing means is a spring braced between the parts.

3. The rod-shortening tool defined in claim 1, further comprising:
an eccentric lever pivotal on one of the parts and bearing through the biasing means on the other of the parts.

4. The rod-shortening tool defined in claim 3, further comprising:
a connecting rod having one end secured to the lever and an opposite end connected through the biasing means with the other part.

5. The rod-shortening tool defined in claim 1, further comprising:
means in one of the housing parts for assisting breaking of the rod along a score line formed by the cutter.

6. The rod-shortening tool defined in claim 5, wherein the means for assisting is formed as at least one bore in the one part in which the rod is snugly engageable, whereby when engaged in the bore the rod can be bent relative to the housing to fracture the rod at the score line.

7. The rod-shortening tool defined in claim 1, wherein one of the housing parts is provided with a handle.

8. The rod-shortening tool defined in claim 1, wherein the cutter is a sharp-edged cutter wheel pivoted on the base part.

9. A tool for shortening an implant rod, the tool comprising:
a base housing part and a movable housing part shiftable toward and away from each other only in a predetermined direction;
a pair of guide rollers rotatable on the movable housing part about respective parallel axes;
a sharp-edge cutter wheel on the base housing part, the rod being fittable between the cutter wheel and the guide rollers in a spread position of the housing parts;
a connecting rod extending through both housing parts;
a compression spring braced between one end of the connecting rod and one of the housing parts and normally urging the one housing part toward the other housing part; and
an eccentric lever coupled to an opposite end of the connecting rod and pivotal on the housing between a cutting position pulling the one end of the connecting rod against the spring and toward the lever to compress the spring and urge the parts together with a predetermined spring force and a releasing position shifting the one end of the connecting rod away from the spring to spread the parts and allow the implant rod to be fitted between the guide rollers and the cutting wheel.

10. A method of circumferentially scoring an implant rod comprising the steps of:
bracing the implant rod between at least one guide roller rotatable on a movable housing part and at least one cutter carried on a base housing part;
biasing the housing parts together with a predetermined constant spring force while relatively rotating the implant rod and the housing parts such that the roller travels around the rod while scoring the implant rod circumferentially.

11. The method defined in claim 10, further comprising the steps before bracing the implant of sequentially:
spreading the housing parts;
fitting the implant rod between the guide wheel and the cutter; and
moving the housing parts back together and thereby loading a spring that applies the spring force during scoring.

12. The method defined in claim 10, further comprising the step after scoring the implant rod of:
fitting the rod to a seat in one of the housing parts; and
breaking the rod at the score line while fitted to the seat.
